(19) United States
(12) Patent Application Publication
(10) Pub. No.: US 2021/0000445 A1
(43) Pub. Date: Jan. 7, 2021

(54) SYSTEMS AND METHODS FOR AUTOMATED BODY SCANNING

(71) Applicant: General Electric Company, Schenectady, NY (US)

(72) Inventors: Huan Tan, Pasadena, CA (US); Bo Wang, Brea, CA (US); Heather Chan, Niskayuna, NY (US); David Martin Mills, Niskayuna, NY (US); Aaron Mark Dentinger, Latham, NY (US); David Andrew Shoudy, Niskayuna, NY (US); Steven Robert Gray, Niskayuna, NY (US); John Robert Hoare, Latham, NY (US)

(21) Appl. No.: 16/458,487

(22) Filed: Jul. 1, 2019

(51) Int. Cl. A61B 8/00; G01S 7/52; G01S 15/89; A61B 8/08

(52) U.S. Cl. CPC A61B 8/4218; G01S 7/52079; A61B 8/4263; A61B 8/483; G01S 15/8906

(57) ABSTRACT

A robotic body scanning system includes a robotic manipulator, a force sensor, a probe, a surface sensing system, and a computing device. The probe is attached to the robotic manipulator and configured to scan the portion of the human body. The surface sensing system is configured to detect a surface of the portion of the human body and generate data representing the portion of the human body. The computing device is configured to receive data representing the portion of the human body from said surface sensing system and generate two or three-dimensional representations of the portion of the human body. The computing device includes a trajectory generation module configured to generate an adapted trajectory for the probe to follow based on the two or three-dimensional representations. The robotic manipulator is configured to move the probe along the adapted trajectory along the portion of the human body.

### STATEMENT REGARDING FEDERALLY SPONSORED RESEARCH AND DEVELOPMENT

This invention was made with Government support under contract number 300455109 awarded by the National Institutes of Health. The Government has certain rights in this invention.

### BACKGROUND

The field of the disclosure relates generally to a system for robotic manipulation of sensors and, more particularly, to an automated system for scanning a patient with a contact sensor.

Three-dimensional information of portions of the human body assists in the diagnosis and treatment of many diseases. For example, three-dimensional information of the human hand may assist in the diagnosis and treatment of at least some diseases afflicting the human hand. At least some current three-dimensional imaging systems use probes and/or sensors, such as ultrasound probes, which physically contact the human body in order to obtain a two and/or three-dimensional images of the body. Typically, a sonographer manually scans portions of the human body with the probes and/or sensors. However, manually scanning portions of the human body is time consuming, labor intensive, and may produce inconsistent results. For example, the three-dimensional images and associated data may depend on the orientation of the probes and/or sensors during the scanning process. As such, manually scanning portions of the human body may produce inconsistent data if the sonographer does not consistently scan the human body with the probes and/or sensors in the same orientation.

### BRIEF DESCRIPTION

In one aspect, a robotic body scanning system for scanning at least a portion of a human body is provided. The robotic body scanning system includes a robotic manipulator, a probe, a surface sensing system, and a computing device. The probe is attached to the robotic manipulator and configured to scan the portion of the human body. The surface sensing system is configured to detect a surface of the portion of the human body and generate data representing the portion of the human body. The computing device is configured to receive data representing the portion of the human body from said surface sensing system and generate a two or three-dimensional representation of the portion of the human body. The computing device includes a trajectory generation module configured to generate an adapted trajectory for the probe to follow based on the two or three-dimensional representation of the portion of the human body. The robotic manipulator is configured to move the probe along the adapted trajectory along the portion of the human body.

In another aspect, a method for scanning at least a portion of a human body with a robotic body scanning system is provided. The robotic body scanning system includes a probe, a computing device, a motion controller, and a robotic manipulator. The probe is attached to the robotic manipulator. The method includes sending an adapted trajectory from the computing device to the motion controller. The adapted trajectory is based on a set of task definitions. The method also includes moving the probe along the adapted trajectory using the robotic manipulator. The method further includes scanning the portion of the human body using the probe as the probe moves along the adapted trajectory.

In yet another aspect, a method for generating a motion trajectory for a probe of a robotic scanning system along at least a portion of a human body is provided. The robotic body scanning system includes the probe, a surface sensing system, and a robotic manipulator. The probe is attached to the robotic manipulator. The method includes receiving at least one task from a medical professional. The method also includes acquiring at least one surface profile of the portion of the human body with the surface sensing system. The method further includes identifying at least one planned trajectory based on the at least one task and the at least one surface profile. The method also includes adapting the at least one planned trajectory to an adapted trajectory based on the at least one surface profile. The method further includes sending the adapted trajectory to a motion controller.

### DRAWINGS

These and other features, aspects, and advantages of the present disclosure will become better understood when the following detailed description is read with reference to the accompanying drawings in which like characters represent like parts throughout the drawings, wherein:

FIG. 1 is a perspective view of a robotic hand scanning system;

FIG. 2 is a functional block diagram of the robotic hand scanning system shown in FIG. 1;

FIG. 3 is a flow diagram of a method of planning a motion trajectory of a probe of the robotic hand scanning system shown in FIG. 1;

FIG. 4 is a flow diagram of a method of scanning a human hand using the robotic hand scanning system shown in FIG. 1;

FIG. 5 is a force diagram of a probe of the robotic hand scanning system shown in FIG. 1 when the probe is touching a human hand; and

FIG. 6 is a force diagram of the probe shown in FIG. 5 as a motion controller plans subsequent probe movements.

Unless otherwise indicated, the drawings provided herein are meant to illustrate features of embodiments of this disclosure. These features are believed to be applicable in a wide variety of systems comprising one or more embodiments of this disclosure. As such, the drawings are not meant to include all conventional features known by those of ordinary skill in the art to be required for the practice of the embodiments disclosed herein.

### DETAILED DESCRIPTION

In the following specification and the claims, reference will be made to a number of terms, which shall be defined to have the following meanings.

The singular forms “a”, “an”, and “the” include plural references unless the context clearly dictates otherwise.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about”, “approximately”, and “substantially”, are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

As used herein, the terms “processor” and “computer,” and related terms, e.g., “processing device,” “computing device,” and “controller” are not limited to just those integrated circuits referred to in the art as a computer, but broadly refers to a microcontroller, a microcomputer, an analog computer, a programmable logic controller (PLC), and application specific integrated circuit (ASIC), and other programmable circuits, and these terms are used interchangeably herein. In the embodiments described herein, “memory” may include, but is not limited to, a computer-readable medium, such as a random access memory (RAM), a computer-readable non-volatile medium, such as a flash memory. Alternatively, a floppy disk, a compact disc—read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a touchscreen, a mouse, and a keyboard. Alternatively, other computer peripherals may also be used that may include, for example, but not be limited to, a scanner. Furthermore, in the exemplary embodiment, additional output channels may include, but not be limited to, an operator interface monitor or heads-up display. Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an ASIC, a PLC, a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

Furthermore, as used herein, the term “real-time” refers to at least one of the time of occurrence of the associated events, the time of measurement and collection of predetermined data, the time to process the data, and the time of a system response to the events and the environment. In the embodiments described herein, these activities and events occur substantially instantaneously.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Embodiments described herein provide systems and methods for a robotic body scanning system configured to scan a portion of a human body. The robotic body scanning system includes a robotic manipulator and a probe attached to the robotic manipulator. The probe is configured to scan the human body and generate one-, two, or three-dimensional data that is representative of the human body while the robotic manipulator moves the probe. The generated data is reconstructed by the computing device to form image(s) and/or representation(s) to assist medical professionals in diagnosing and treating diseases. The robotic body scanning system described herein automates the scanning process to accurately capture scanning data and positional data to produce accurate image(s) and/or representation(s). Specifically, the robotic body scanning system may be configured to acquire sonograms which may be used to generate two or three-dimensional images, representations, and/or models of the human body as well as blood-flow sequences showing the blood flow in the portion of the human body that has been scanned. More specifically, the robotic body scanning systems described herein include a contact scanning configuration and a non-contact scanning configuration. The contact scanning configuration scans the portion of the human body by contacting the probe with the portion of the human body that the probe scans, while the non-contact scanning configuration scans the portion of the human body without contacting the probe with the portion of the human body that the probe scans.

Additionally, the robotic body scanning systems described herein include a surface sensing system and a computing device. The computing device may include anatomy recognition and surface extraction software and a database of reference anatomy scan trajectories. In an alternative embodiment, a medical professional manually recognizes the anatomy and defines the anatomy to be scanned within the computing device. The surface sensing system images the portion of the human body to be scanned and sends the captured images to the computing device. The computing device identifies the region of the human body imaged by the surface sensing system and receives at least one task requirement from a medical professional. The computing device generates a trajectory for the probe to follow based on the imaged portion of the human body, the task requirement, and at least one reference trajectory from the database of reference anatomy scan trajectories. The robotic manipulator moves the probe along the trajectory and the probe scans the human body.

Furthermore, the contact scanning configuration performs a force analysis on the probe to ensure that the probe safely contacts and scans the human body. Specifically, the robotic body scanning system includes a force sensor configured to measure a contact force of the probe on the human body. The force sensor sends the measured contact force data to a controller that adjusts the path of the probe based on the measured contact force. Moreover, the force sensor may estimate changes of the human body during the scanning process, such as movements by the patient during the scanning process. As such, the robotic body scanning system is able to adjust a path of the probe based on the measured contact force.

Moreover, the robotic body scanning systems described herein dynamically adapts a trajectory of the probe based on movements of the human body during the scanning process. Specifically, if the human body moves while the robotic body scanning system scans the human body, the surface sensor detects the movement and detects the new position of the human body. The computing device re-computes the adapted trajectory, and adjusts the path of the probe to follow a new trajectory based on the new position of the human body. The computing device is able to re-compute the adapted trajectory based on small adjustments or movements of the human body. If the adjustments or movements are larger than a predetermined threshold, however, the computing device stops the scanning process and generates a new trajectory for the probe to follow based on the imaged portion of the human body, the task requirement, and at least one reference trajectory from the database of reference anatomy scan trajectories. The robotic manipulator moves the probe along the new trajectory and the probe scans the human body.

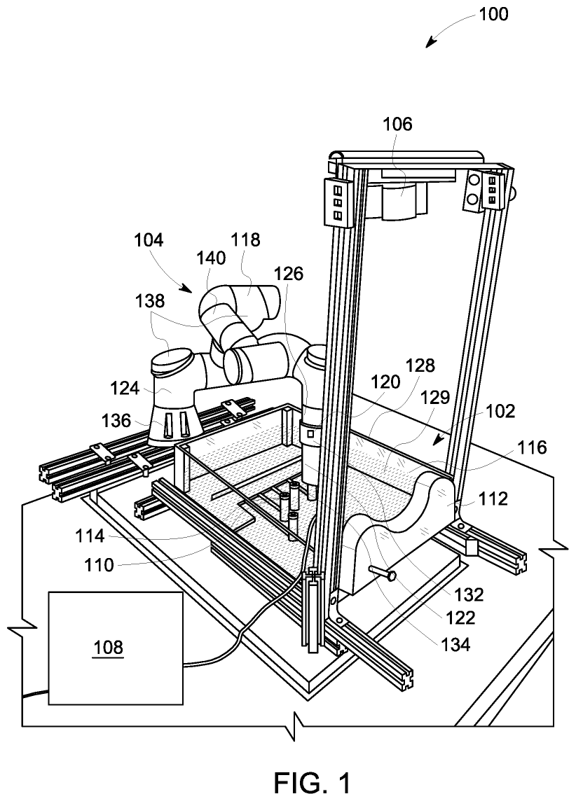

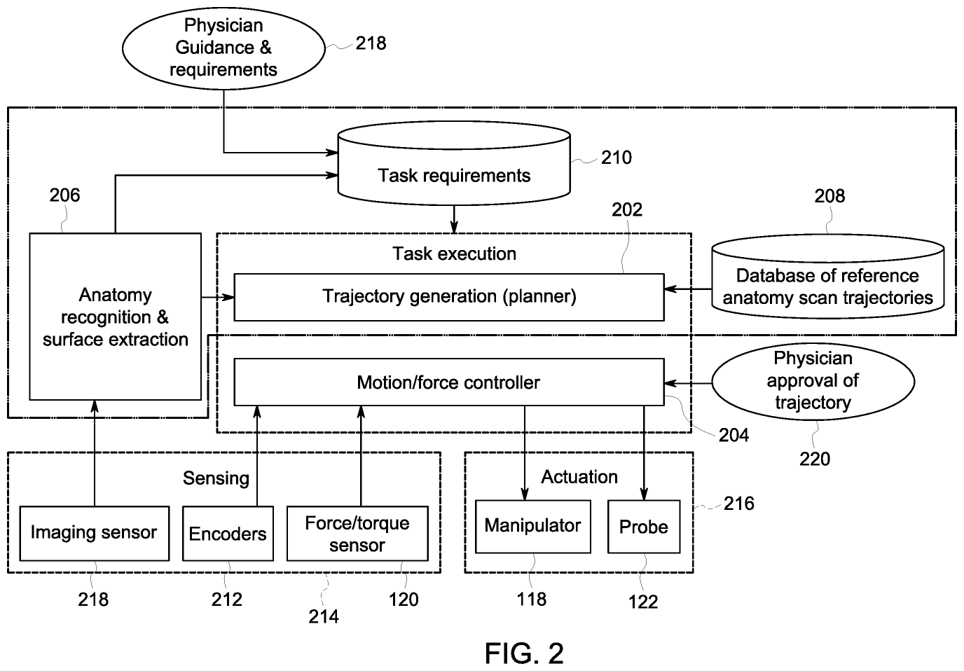

FIG. 1 is a perspective view of a robotic body scanning system 100. FIG. 2 is a functional block diagram of robotic body scanning system 100 shown in FIG. 1. In the illustrated embodiment, scanning system 100 is configured to scan a human hand to provide data to medical professionals for diagnosis and treatment of at least some diseases afflicting the human hand. In alternative embodiments, scanning system 100 is configured to scan any part of the human body. Specifically, scanning system 100 is configured to generate two-dimensional and three-dimensional images and/or representations of the human body that may assist medical professionals with diagnosis and treatment. Scanning system 100 automates the scanning process to accurately capture scanning data and positional data to produce accurate two and three-dimensional images and/or representations, as described herein.

Scanning system 100 includes a positioning system 102, a robotic manipulation system 104, a surface sensor 106, a computing device 108, and a motion controller 204. Positioning system 102 is configured to position the human body within scanning system 100. Positioning system 102 may include a bed configured to position the entire human body relative to robotic manipulation system 104, a positioning device configured to position a specific portion of the human body relative to robotic manipulation system 104, a bath 128 configured to immerse a portion of the human body in a medium 129 and position the portion of the human body relative to robotic manipulation system 104 (which may be particularly useful for the non-contact scanning configuration, but may also be useful for the contact scanning configuration), and/or any other device configured to position at least a portion of the human body relative to robotic manipulation system 104. In the illustrated embodiment, positioning system 102 is configured to position the human hand within scanning system 100 and includes five finger holders 110 and a wrist holder 112 in the exemplary embodiment. Finger holders 110 each define a finger placement indentation 114 configured to receive a finger of the human hand. Wrist holder 112 defines a wrist indentation 116 configured to receive a wrist/forearm of the human arm. Scanning system 100 also includes a surface sensing system 214 and an actuation system 216 (both shown in FIG. 2). Surface sensing system 214 includes surface sensor 106, a plurality of encoders 212, and a force sensor 120, and actuation system 216 includes a robotic manipulator 118 and a probe 122.

In the non-contact scanning configuration, bath 128 is filled with medium 129 that transports sound waves from probe 122 into the human body and back to probe 122. In the exemplary embodiment, medium 129 is water. In alternative embodiments, medium 129 is an ultrasound gel including combinations of propylene glycol, glycerin, perfumes, dyes, polymers, and water. A portion of the human body is submerged in medium 129, and robotic manipulator 118 is configured to position probe 122 proximate, but not contacting, the human body and move probe 122 along the human body to scan the human body. In the exemplary embodiment, probe 122 is an ultrasound probe configured to generate ultrasound data and/or ultrasound images of the body. However, probe 122 may be any sensing device that enables scanning system 100 to operate as described herein.

Robotic manipulation system 104 includes robotic manipulator 118, force sensor 120, and probe 122. In the exemplary embodiment, robotic manipulator 118 is a robotic arm configured to move probe 122 along the human body. Robotic manipulator 118 is communicatively coupled with motion controller 204. Specifically, in the contact scanning configuration, robotic manipulator 118 is configured to position probe 122 on the human body with an appropriate amount of force and move probe 122 along the human body to scan the human body. Force sensor 120 is a sensor configured to measure a contact force that probe 122 places on the body. In the exemplary embodiment, probe 122 is an ultrasound probe configured to generate ultrasound data and/or ultrasound images of the body. In the contact scanning configuration, a layer of medium 129 that transports sound waves from probe 122 into the human body and back to probe 122 is applied to probe 122. Probe 122 may be any sensing device that enables scanning system 100 to operate as described herein.

Robotic manipulator 118 includes a first end 124 and an opposite second end 126, and probe 122 includes a first end 132 and an opposite second end 134. First end 124 of robotic manipulator 118 is attached to a base 136, and second end 126 of robotic manipulator 118 is attached to force sensor 120. Force sensor 120 is attached to first end 132 of probe 122. As such, robotic manipulator 118 is configured to move force sensor 120 and probe 122.

Robotic manipulator 118 also includes a plurality of joints 138 and a plurality of links 140 that connect joints 138. Links 140 are typically rigid connecting units that connect joints 138. In the exemplary embodiment, links 140 are hollow metal tubes that connect joints 138 and that maintain a position of force sensor 120 and probe 122. Joints 138 are typically actuation devices configured to move links 140 through a predetermined path. Specifically, joints 138 may include linear joints, rotational joints, orthogonal joints, twisting joints, and/or revolving joints. However, joints 138 may be any type of joint that enables scanning system 100 to operate as described herein. Linear joints typically slide a first link in a direction parallel to an orientation of a second link. Rotational joints typically rotate a first link about a second link. Orthogonal joints typically slide a first link in a direction parallel to an orientation of a second link, but, unlike a linear joint, the first link has an orientation that is orthogonal to the orientation of the second link. Twisting joints typically twist a first link relative to a second link with the same orientation as the first link. Revolving joints typically twist a first link relative to a second link that has an orientation that is orthogonal to the first link. Robotic manipulator 118 includes a plurality of joints 138 and links 140 attached to each other and arranged such that joints 138 and links 140 cooperate to move probe 122 in a smooth motion over a body.

Moreover, in the exemplary embodiment, robotic manipulator 118 includes a plurality of encoders 212 each configured to determine a location of an assembled portion of robotic manipulator 118. Encoders 212 are electrical and mechanical devices each configured to detect a position and/or orientation of an assembled portion of robotic manipulator 118. Encoders 212 are communicatively coupled to motion controller 204. In the exemplary embodiment, each joint 138 includes an encoder 212 configured to mechanically detect a position and/or orientation of joint 138, convert the mechanically detected position and/or orientation into an electrical signal, and transmit the electrical signal to motion controller 204. Computer device 108 then controls robotic manipulator 118 based at least in part on the received signals from encoders 212. Robotic manipulator 118 may also include other location/orientation detecting devices including, for example and without limitation, a global positioning system (GPS) device, an inertial measurement unit (IMU), a light detection and ranging (LIDAR) device, a camera, an infrared device, an eddy current sensor, a sonar device, a radar device, and/or any other positioning sensor. In alternative embodiments, robotic manipulator 118 includes any localization sensor that enables scanning system 100 to operate as described herein.

Force sensor 120 is configured to measure the contact force of probe 122 on the human body. Force sensor 120 is an electrical device including a load cell (not shown) or a transducer configured to generate an electrical signal whose magnitude is directly proportional to a force being measured. Force sensor 120 is communicatively coupled with motion controller 204 and robotic manipulator 118. Force sensor 120 transmits the electrical signal to computer device 108 and/or a controller. Computer device 108 then controls robotic manipulator 118 based at least in part on the electrical signal received from force sensor 120. Additionally, force sensor 120 may transmit the electrical signal directly to robotic manipulator 118 to prevent the contact force from rising above a predetermined threshold.

In the exemplary embodiment, probe 122 is an ultrasound probe configured to transmit ultrasound waves (i.e., sound waves with frequencies higher than frequencies that are audible to humans) into internal body structures. The ultrasound waves reflect off internal tissue, and different types of tissues reflect different magnitudes of sound waves back to the ultrasound probe. The reflected waves are recorded and processed to generate a two or three-dimensional image and/or representation of the body. As used herein, a scan is an ultrasound image generated by probe 122. Computing device 108 receives the scan from probe 122 and controls robotic manipulator 118 based at least in part on the scans from probe 122. Probe 122 may be any type of ultrasound probe that enables scanning system 100 to operate as described herein. Additionally, probe 122 is not limited to ultrasound probes. Rather, probe 122 may be any type of sensor that enables scanning system 100 to operate as described herein.

Surface sensor 106 is positioned above scanning system 100 and is configured to acquire at least one surface profile of a portion of the human body. The surface profile includes images and/or other sensor data of the surface of the human body. As used herein, a surface profile from surface sensor 106 may be an image, detected surface, outline, model, or representation of the surface of the human body. Additionally, as used herein, an image or detected surface from surface sensor 106 may be an optical image and/or video acquired using an imaging sensor included with surface sensor 106, or it may be a representative outline of the surface of interest of the human body. In the exemplary embodiment, surface sensor 106 includes an RGBD sensor including an optical camera configured to detect light visible with the human eye and a depth sensor configured to detect infrared radiation. However, surface sensor 106 may be any type of sensor that enables scanning system 100 to operate as described herein, including, without limitation, an infrared camera, a stereo RGB camera, a camera with a Light Detection and Ranging (LIDAR) sensor, a sonar device (e.g., air ultrasound), or a camera with a time-of-flight sensor. Surface sensor 106 may also be a two-dimensional sensor that captures the outline of the human body, such as an RGB camera. A depth from surface sensor 106 to the human body may either be detected by surface sensor 106, acquired by a separate sensor, or manually entered by an operator. Surface sensor 106 is communicatively coupled with computing device 108. Surface sensor 106 transmits the images of the body to computing device 108, and computing device 108 controls robotic manipulator 118 based at least in part on the images from surface sensor 106.

Computing device 108 includes a computer system that includes at least one processing device (not shown in FIG. 1) and at least one memory device (not shown in FIG. 1) that executes executable instructions to control scanning system 100 and generate scanning data for the human body. Computing device 108 includes, for example, image processing software 206 or other analytic software configured to analyze images acquired from surface sensor 106. Computing device 108 may also include a database 208 communicatively coupled to a trajectory generation module 202 and a task requirements module 210. Database 208 is a database of reference anatomy scan trajectories. Task requirements module 210 contains a predetermined set of requirements established by a medical professional. For example, the medical professional may specify a predetermined region on the body to be scanned or a predetermined set of movements for probe 122 to follow along a surface of the body. More specifically, task requirements module 210 may receive a set of predetermined requirements from a physician guidance and requirements module 218 in which a medical professional has entered the set of predetermined requirements. In the exemplary embodiment, computing device 108 is also configured to operate as a data acquisition device to receive data from probe 122. In one embodiment, for example, computing device 108 receives and processes scans from probe 122. Computing device 108 stores and analyzes the scans, which are used to facilitate diagnosis and treatment of diseases afflicting the body. In another embodiment, for example, computing device 108 receives and processes the images from surface sensor 106, which, with the scans from probe 122, are used to create two or three-dimensional representations of the body.

In yet another embodiment, computing device 108 receives and processes images from surface sensor 106 and receives instructions sent by a medical professional. Computing device 108 then plots a planned trajectory and adapts the planned trajectory using reference anatomy scan trajectories from database 208. Specifically, the planned trajectory is generated by trajectory generation module 202 based on input from task requirements module 210 and database 208. More specifically, task requirements module 210 receives a task from physician guidance and requirements module 218 which specifies a certain portion of the human body. Trajectory generation module 202 receives a reference anatomy scan trajectory of the specified portion of the human body, which is a model trajectory for probe 122 to follow over the specified portion of the human body. Because each individual patient may have slightly different anatomy, the reference anatomy scan trajectory of the specified portion of the human body must be adjusted to conform to the specific anatomy of the patient. As such, trajectory generation module 202 of computing device 108 is then configured to adjust the planned trajectory to an adapted trajectory based on the images acquired by surface sensor 106 to adapt the planned trajectory to the specific anatomy of the patient positioned within scanning system 100. The adapted trajectory is then sent to motion controller 204 which controls robotic manipulator 118 to follow the planned trajectory.

In the exemplary embodiment, trajectory generation module 202 is communicatively coupled to task requirements module 210, image processing software 206, database 208, and motion controller 204 and is configured to at least partially control motion controller 204. For example, trajectory generation module 202 receives information from task requirements module 210, database 208, and image processing software 206 and controls motion controller 204 based on the received information. Specifically, trajectory generation module 202 receives a set of task requirements from task requirements module 210 and compares the task requirements to the data being analyzed by image processing software 206 and reference anatomy scan trajectories from database 208. Trajectory generation module 202 controls motion controller 204 based on the data received from task requirements module 210, database 208, and image processing software 206. Specifically, trajectory generation module 202 sends the adapted trajectory to motion controller 204.

In the exemplary embodiment, motion controller 204 is communicatively coupled to trajectory generation module 202, encoders 212, force sensor 120, robotic manipulator 118, probe 122, and a physician approval module 220, and is configured to control robotic manipulator 118 and probe 122. Specifically, motion controller 204 receives data from trajectory generation module 202, encoders 212, force sensor 120, and physician approval module 220 and sends control data to robotic manipulator 118 and probe 122. More specifically, motion controller 204 receives the adapted trajectory from trajectory generation module 202, position/orientation data of robotic manipulator 118 from encoders 212, the contact force from force sensor 120, and approval for the planned trajectory from physician approval module 220. Motion controller 204 adjusts the adapted trajectory to an adjusted trajectory based on the contact force, position/orientation data of robotic manipulator 118, and the scanning data. Motion controller 204 controls robotic manipulator 118 and probe 122 to follow the adapted trajectory. Additionally, motion controller 204 controls robotic manipulator 118 and probe 122 based on input from force sensor 120 and encoders 212.

During operation, at least a portion of the human body is placed in scanning system 100. Specifically, in the illustrated embodiment, scanning system 100 is a hand scanning system, with the fingers of the hand placed in finger holders 110 and the wrist placed in wrist holder 112. In at least the non-contact scanning configuration bath 128 includes medium 129, and the portion of the human body is submerged in medium 129. In contrast, in the contact scanning configuration, scanning system 100 may not include bath 128. Rather, medium 129 may be applied to second end 134 of probe 122 or the human body, and second end 134 of probe 122 contacts the human body. Next, surface sensor 106 images the hand and sends the acquired images to computing device 108 and image processing software 206. Computing device 108 then analyzes the images and sends the analyzed images to trajectory generation module 202. Trajectory generation module 202 simultaneously receives predetermined task requirements, such as a predetermined region on the body to be scanned (e.g., in the illustrated embodiment, the hand) from task requirements module 210 and reference anatomy scan trajectories from database 208, and determines a planned trajectory for probe 122 to follow across the hand based on the reference anatomy scan trajectories from database 208 and the task requirements. Trajectory generation module 202 of computing device 108 then adjusts the planned trajectory to the adapted trajectory based on the images acquired by surface sensor 106 to adapt the planned trajectory to the specific anatomy of the patient positioned within scanning system 100. Trajectory generation module 202 then transmits the adapted trajectory to motion controller 204. After motion controller 204 receives the adapted trajectory from trajectory generation module 202, motion controller 204 moves probe 122 to a starting point of the adapted trajectory. Motion controller 204 then moves probe 122 along the adapted trajectory, and probe 122 scans the body and sends the scan data to computing device 108. In the non-contact scanning configuration, probe 122 is moved over the surface of the human body and does not contact the human body. In the contact scanning configuration, probe 122 contacts the human body, and force sensor 120 simultaneously determines the contact force of probe 122 on the body and sends the force data to motion controller 204. During the scanning process, encoders 212 are continually monitoring the position/orientation of joints 138 and links 140 within robotic manipulator 118 and sending the position/orientation data to motion controller 204. Motion controller 204 continually adjusts the adapted trajectory based on the data received from encoders 212, force sensor 120, task controller 202, and image processing software 206 to an adjusted trajectory. Motion controller 204 moves probe 122 along the adjusted trajectory until the scan is complete.

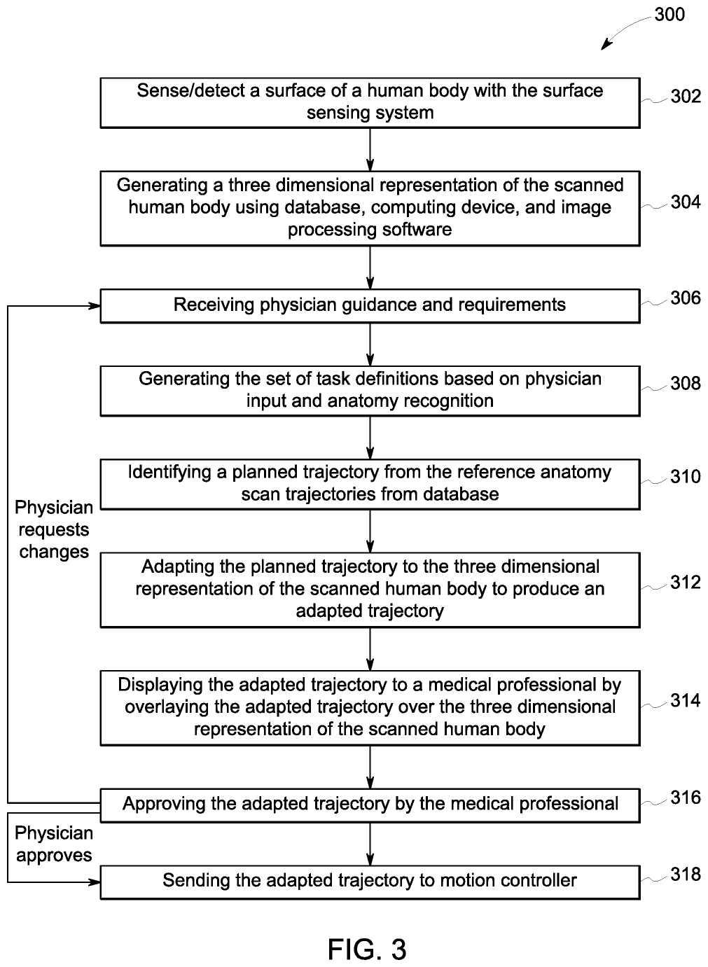

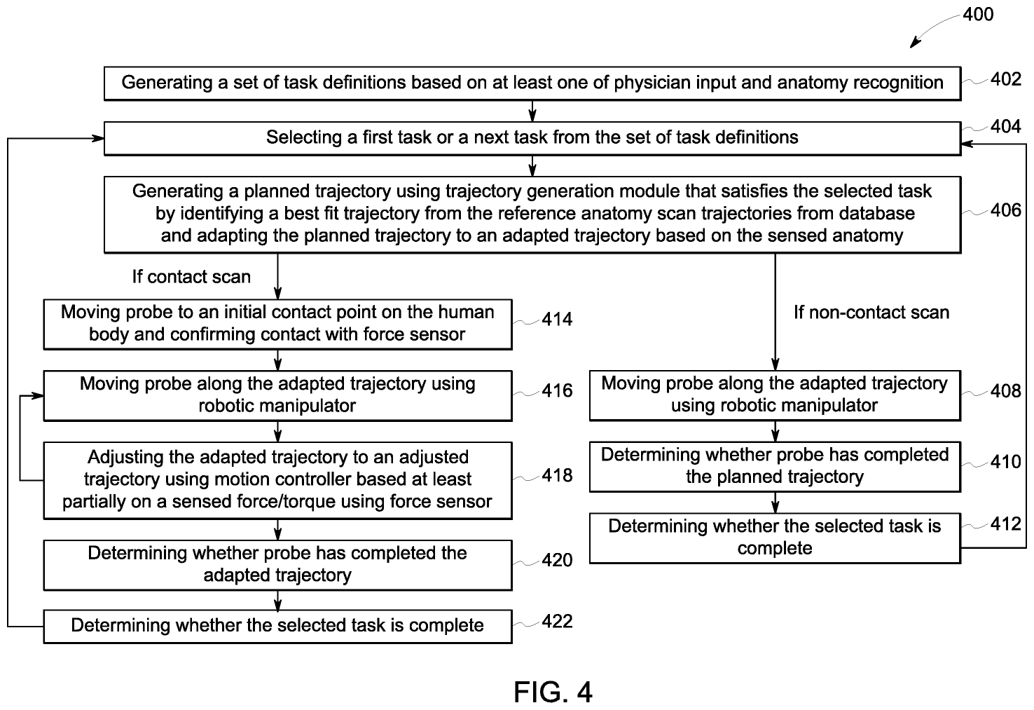

FIG. 3 is a flow diagram of a method 300 of generating a planned trajectory and an adapted trajectory of probe 122 of scanning system 100 and sending the adapted trajectory of probe 122 to motion controller 204. Specifically, method 300 is a method of generating 406 the planned trajectory and adapting the planned trajectory to the adapted trajectory as shown in FIG. 4. Method 300 includes scanning 302 the human body with surface sensor 106. Method 300 also includes generating 304 a two or three-dimensional representation of the scanned human body using database 208, computing device 108, and image processing software 206. Method 300 further includes receiving 306 physician guidance and requirements. Method 300 also includes generating 308 the set of task definitions based on physician input and/or anatomy recognition. The task definition may be generated 308 based on, for example, manual input from the physician and/or a computer-based automatic recognition. Method 300 further includes identifying 310 a planned trajectory from the reference anatomy scan trajectories from database 208. Method 300 also includes adapting 312 the planned trajectory to the two or three-dimensional representation of the scanned human body to produce an adapted trajectory. Method 300 further includes displaying 314 the adapted trajectory to a medical professional by overlaying the adapted trajectory on the two or three-dimensional representation of the scanned human body. Method 300 also includes approving 316 the adapted trajectory by the medical professional. If the medical professional requests changes to the adapted trajectory, steps 306 to 316 are repeated until the medical professional approves the adapted trajectory. Method further includes sending 318 the adapted trajectory to motion controller 204.

FIG. 4 is a flow diagram of a method 400 of scanning a hand using scanning system 100. Method 400 includes generating 402 a set of task definitions based on at least one of physician input and anatomy recognition. Method 400 also includes selecting 404 a first task or a next task from the set of task definitions. Method 400 further includes generating 406 a planned trajectory using trajectory generation module 202 that satisfies the selected task by identifying a best fit trajectory from the reference anatomy scan trajectories from database 208 and adapting the planned trajectory to an adapted trajectory based on the sensed anatomy. In the non-contact scanning configuration, method 400 also includes moving 408 probe 122 along the adapted trajectory using robotic manipulator 118. Method 400 further includes determining 410 whether probe 122 has completed the adapted trajectory. Method 400 also includes determining 412 whether the selected task is complete. If the selected task is complete, steps 402-412 are repeated until the all task requirements are satisfied. In the contact scanning configuration, method 400 further includes moving 414 probe 122 to an initial contact point on the human body and confirming contact with force sensor 120. Method 400 also includes moving 416 probe 122 along the adapted trajectory using robotic manipulator 118. Method 400 further includes adjusting 418 the adapted trajectory to an adjusted trajectory using motion controller 204 based at least partially on a sensed force using force sensor 120. Method 400 further includes determining 420 whether probe 122 has completed the adapted trajectory. Method 400 also includes determining 422 whether the selected task is complete. If the task requirements are not satisfied, steps 402-422 are repeated until the task requirements are satisfied.

As such, the images acquired by surface sensor 106 are used by image processing software 206, database 208, and computing device 108 to generate the planned trajectory and the adapted trajectory for probe 122 to follow. More specifically, a two or three-dimensional representation of the scanned human body is generated using computing device 108, image processing software 206, and the images acquired by surface sensor 106. Next, a planned trajectory is generated using reference anatomy scan trajectories from database 208, computing device 108, image processing software 206, and the images acquired by surface sensor 106. After the planned trajectory is generated, an adapted trajectory is generated by adapting the planned trajectory to the two or three-dimensional representation of the scanned human body using the computing device 108, image processing software 206, the images acquired by surface sensor 106, and the reference anatomy scan trajectories from database 208. Once a medical professional, such as a physician, a technician, a nurse, or any other medical professional, has approved the adapted trajectory, the adapted trajectory is sent to motion controller 204 and motion controller 204 adjusts the adapted trajectory based on the measured contact force from force sensor 120. In an alternative embodiment, the medical professional manually adjusts the adapted trajectory before the adapted trajectory is sent to motion controller 204. The medical professional then approves the adjusted adapted trajectory which is then sent to motion controller 204. Accordingly, surface sensor 106 and force sensor 120 are used to safely scan the body with predictive planning and control of the scanning process.

Additionally, the two or three-dimensional images acquired by scanning system 100 and/or the two or three-dimensional representations generated by computing device 108 may be used to further adapt and/or adjust the adapted trajectory and/or generate a new planned trajectory. For example, image processing software 206 may be configured to analyze the two or three-dimensional images and/or representations in real time to determine if scanning system 100 is accurately scanning the sensed anatomy. Specifically, image processing software 206 may include artificial intelligence and/or conventional image processing capabilities that enable image processing software 206 to analyze the two or three-dimensional images and/or representations in real time. Image processing software 206 may include algorithms that search the two or three-dimensional images and/or representations for the sensed anatomy and/or key features of the sensed anatomy. If image processing software 206 determines that the sensed anatomy and/or the key features of the sensed anatomy are present within a predetermined range within the two or three-dimensional images and/or representations, then probe 122 continues scanning the sensed anatomy along the adapted trajectory. If, however, image processing software 206 determines that the sensed anatomy and/or the key features of the sensed anatomy are outside the predetermined range within the two or three-dimensional images and/or representations, the adapted trajectory is adjusted by computing device 108 to maintain the sensed anatomy and/or the key features of the sensed anatomy within the predetermined range during the scanning process. Moreover, if image processing software 206 determines that the sensed anatomy is not within the two or three-dimensional images and/or representations, and/or computing device 108 cannot adjust the adapted trajectory to maintain the sensed anatomy and/or the key features of the sensed anatomy within the predetermined range, then scanning system 100 may initiate an anatomy search mode. In the anatomy search mode, robotic manipulator 118 and probe 122 scan within a region on a surface of the human body where the sensed anatomy was last detected until the sensed anatomy is detected and two or three-dimensional data is acquired to re-compute the adapted trajectory and finish the scan. If, however, the imaging processing software 206 determines that the sensed anatomy is partially outside the predetermined range, robotic manipulator 118 and probe 122 will return to a location where the sensed anatomy was within the predetermined range and the adapted trajectory will be adjusted by the computing device 108 to maintain the sensed anatomy within the predetermined range.

Furthermore, scanning system 100 may be controlled by image processing software 206 in a closed feedback loop that adjusts and/or adapts the scan parameters of probe 122 based on the two or three-dimensional images and/or representations. For example, image processing software 206 may be configured to control a depth parameter of probe 122. If the sensed anatomy is deeper within the human body than anticipated by computing device 108, image processing software 206 may be configured to adjust the depth parameter of probe 122 to acquire accurate images of the sensed anatomy. Furthermore, if a quality of the two or three-dimensional images does not meet or exceed a predetermined image quality parameter, image processing software 206 may be configured to adjust the scan parameters of probe 122 to acquire accurate images of the sensed anatomy.

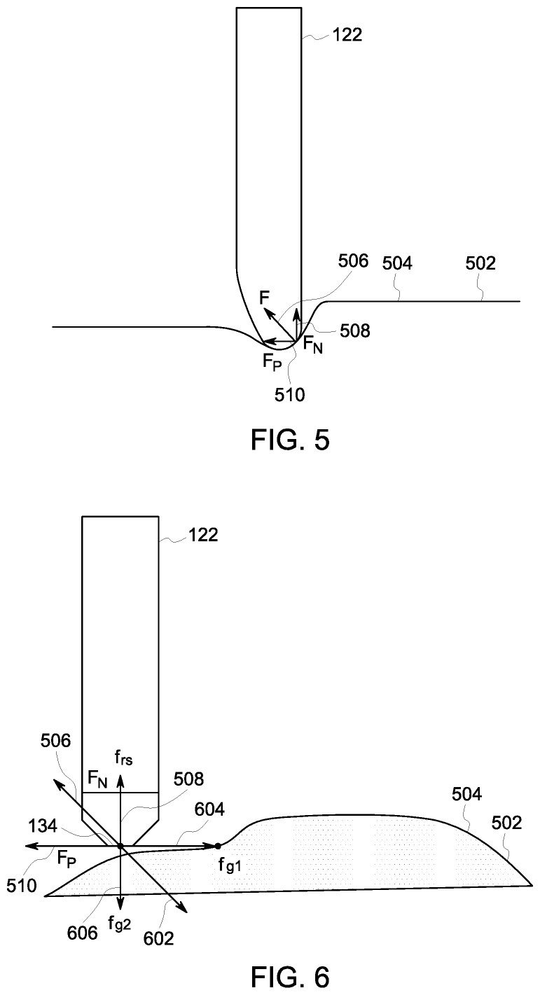

FIG. 5 is a force diagram of probe 122 of scanning system 100 shown in FIG. 1 when probe 122 contacts a portion of a human body 502 in the contact scanning configuration. As shown in FIG. 5, second end 134 of probe 122 contacts a surface 504 of human body 502 which causes a reaction force 506 on probe 122. Reaction force 506 includes a normal component 508 oriented normal to surface 504 of human body 502 and a parallel component 510 oriented parallel to surface 504 of human body 502.

In the exemplary embodiment, force sensor 120 is a six-axis force sensor that measures forces in the x, y, and z directions. Specifically, force sensor 120 measures forces 506 and 508. In the exemplary embodiment, the magnitude of normal component 508 is determined, at least in part, by the amount of normal force required to acquire an ultrasound image. Normal component 508 may also be used as a safety signal by force sensor 120 and motion controller 204. Specifically, if normal component 508 is greater than a predetermined threshold, force sensor 120 stops robotic manipulator 118. If normal component 508 is not equal to the desired amount of normal force specified in the adapted trajectory, normal component 508 is used by motion controller 204 to correct the applied force.

FIG. 6 is a force diagram of probe 122 of scanning system 100 shown in FIG. 1 and surface 504 of human body 502 that illustrates the forces necessary to safely move probe 122 along surface 504 of human body 502. Specifically, motion controller 204 performs a force analysis on probe 122. Motion controller 204 then uses the results of the force analysis, along with methods 300 and 400 described above, to adjust the adapted trajectory. As shown in FIG. 6, a desired force 602 is the force of probe 122 exerted on surface 504 of human body 502. Desired force 602 includes a parallel component 604 oriented parallel to surface 504 of human body 502 and is the force needed to move probe 122 along surface 504 of human body 502. Desired force 602 also includes a normal component 606 oriented normal to surface 504 of human body 502 and is the force that is exerted on surface 504 of human body 502 to produce an accurate scan. Reaction force 506 is the reaction to desired force 602 and is oriented opposite desired force 602. As such, parallel component 510 of reaction force 506 is the reaction to parallel component 604 of desired force 602 and is oriented opposite parallel component 604 of desired force 602. Similarly, normal component 508 of reaction force 506 is the reaction to normal component 606 of desired force 602 and is oriented opposite normal component 606 of desired force 602. Normal component 606 of desired force 602 is determined by the task requirements provided by the medical professional and may be determined by the type of scan requested by the medical professional. Parallel component 604 of desired force 602 is the force required to overcome friction on surface 504 of human body 502 and to follow the adapted trajectory. When probe 122 is traveling along the adapted trajectory at a constant velocity, the net force, desired force 602 plus reaction force 506, is zero.

Force sensor 120 sends reaction force 506 data to motion controller 204 which adjusts the adapted trajectory based on reaction force 506. Moreover, force sensor 120 may be configured to estimate changes of the human body, such as movements by the body, during the scanning process based on reaction force 506.

Embodiments described herein provide systems and methods for a robotic body scanning system configured to scan a portion of a human body. The robotic body scanning system includes a robotic manipulator and a probe attached to the robotic manipulator. The probe is configured to scan the human body and generate a three-dimensional representation of the human body to assist medical professionals in diagnosing and treating diseases. The robotic body scanning system described herein automates the scanning process to accurately capture scanning data and positional data to produce an accurate three-dimensional representation. More specifically, the robotic body scanning systems described herein include a contact scanning configuration and a non-contact scanning configuration. The contact scanning configuration scans the portion of the human body by contacting the portion of the human body that the probe scans with the probe, while the non-contact scanning configuration scans the portion of the human body without the probe contacting the portion of the human body that the probe scans.

Additionally, the robotic body scanning systems described herein include a surface sensing system and a computing device. The computing device may include anatomy recognition and surface extraction software and a database of reference anatomy scan trajectories. The surface sensing system images the portion of the human body to be scanned and sends the captured images to the computing device. The computing device identifies the region of the human body imaged by the surface sensing system and receives at least one task requirement from a medical professional. The computing device generates a trajectory for the probe to follow based on the imaged portion of the human body, the task requirement, and at least one reference trajectory from the database of reference anatomy scan trajectories. The robotic manipulator moves the probe along the trajectory and the probe scans the human body.

Furthermore, the contact scanning configuration performs a force analysis on the probe to ensure that the probe safely contacts and scans the human body. Specifically, the robotic body scanning system includes a force sensor configured to measure a contact force of the probe on the human body. The force sensor sends the measured contact force data to a controller that adjusts the path of the probe based on the measured contact force. Moreover, the force sensor may estimate changes of the human body during the scanning process, such as movements by the patient during the scanning process. As such, the robotic body scanning system is able to adjust a path of the probe based on the measured contact force.

An exemplary technical effect of the methods, systems, and apparatus described herein includes at least one of: (a) moving a probe across a human body; (b) scanning a human body with the probe; and (c) generating scanning data of a human body.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

Exemplary embodiments of methods, systems, and apparatus for scanning are not limited to the specific embodiments described herein, but rather, components of systems and/or steps of the methods may be utilized independently and separately from other components and/or steps described herein. For example, the methods, systems, and apparatus may also be used in combination with other scanning systems, and are not limited to practice with only the systems and methods as described herein. Rather, the exemplary embodiment can be implemented and utilized in connection with many other applications, equipment, and systems that may benefit from automated scanning systems.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to disclose the embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A robotic body scanning system for scanning at least a portion of a human body, said robotic body scanning system comprising:
a robotic manipulator;
a probe attached to said robotic manipulator and configured to scan the portion of the human body;
a surface sensing system configured to detect a surface of the portion of the human body and generate data representing the portion of the human body; and
a computing device configured to receive the data representing the portion of the human body from said surface sensing system and generate a two or three-dimensional representation of the portion of the human body, said computing device including a trajectory generation module configured to generate an adapted trajectory for said probe to follow based on the two or three-dimensional representation of the portion of the human body, wherein said robotic manipulator is configured to move said probe along the adapted trajectory along the portion of the human body.

2. The robotic body scanning system in accordance with claim 1, wherein said probe comprises an ultrasound probe.

3. The robotic body scanning system in accordance with claim 1, wherein said robotic manipulator comprises a robotic arm comprising a plurality of links and joints attached to each other and arranged such that said plurality of links and joints cooperate to move said probe along a planned trajectory on the portion of the human body.

4. The robotic body scanning system in accordance with claim 1, wherein said robotic manipulator comprises at least one encoder configured to detect and record a position of at least one of said robotic manipulator and said probe along the adapted trajectory on the portion of the human body.

5. The robotic body scanning system in accordance with claim 1 further comprising a motion controller configured to move said robotic manipulator and said probe along a planned trajectory on the portion of the human body.

6. The robotic body scanning system in accordance with claim 5 further comprising a force sensor attached to said robotic manipulator and said probe and configured to detect a contact force of said probe on the portion of the human body.

7. The robotic body scanning system in accordance with claim 6, wherein said motion controller is configured to adjust the adapted trajectory to an adjusted trajectory based on signals received from said force sensor.

8. The robotic body scanning system in accordance with claim 7, wherein said robotic manipulator comprises at least one encoder configured to detect and record a position of at least one of said robotic manipulator, said force sensor, and said probe along the adapted trajectory on the portion of the human body, and wherein said motion controller is configured to control said robotic manipulator and said probe based on signals received from said force sensor and said at least one encoder.

9. The robotic body scanning system in accordance with claim 6, wherein said force sensor is configured to directly control said robotic manipulator when the contact force is greater than a predetermined threshold to protect the portion of the human body.

10. The robotic body scanning system in accordance with claim 6, wherein said computing device is configured to receive a plurality of task requirements, and wherein said motion controller is configured to control said robotic manipulator and said probe based on the plurality of task requirements.

11. The robotic body scanning system in accordance with claim 1, wherein said computing device is configured to analyze the two or three-dimensional representation of the portion of the human body to determine if a sensed anatomy is within a predetermined range.

12. The robotic body scanning system in accordance with claim 11, wherein said computing device is configured to adjust the adapted trajectory to maintain the sensed anatomy within the predetermined range.

13. A method for scanning at least a portion of a human body with a robotic body scanning system including a probe, a computing device, a motion controller, and a robotic manipulator, wherein the probe is attached to the robotic manipulator, said method comprising:
sending an adapted trajectory from the computing device to the motion controller, wherein the adapted trajectory is based on a set of task definitions;
moving the probe along the adapted trajectory using the robotic manipulator; and
scanning the portion of the human body using the probe as the probe moves along the adapted trajectory.

14. The method in accordance with claim 13 further comprising:
generating the set of task definitions based on at least one task from a medical professional; and
selecting a first task or a next task from the set of task definitions.

15. The method in accordance with claim 14 further comprising:
generating a planned trajectory that satisfies the selected task by identifying a best fit trajectory from a plurality of reference anatomy scan trajectories;
acquiring at least one surface profile of the portion of the human body using a surface sensing system; and
adapting the planned trajectory to the adapted trajectory based on the at least one surface profile acquired by the surface sensing system.

16. The method in accordance with claim 15 further comprising:
moving the probe to an initial contact point on the portion of the human body;
contacting the initial contact point with the probe; and
confirming the contact with a force sensor.

17. The method in accordance with claim 16 further comprising detecting a position of at least one of the robotic manipulator and the probe along the planned trajectory on the portion of the human body using at least one encoder.

18. The method in accordance with claim 16, further comprising:
detecting a contact force of the probe on the portion of the human body using the force sensor; and
adjusting the adapted trajectory to an adjusted trajectory based at least partially on the contact force.

19. A method for generating a motion trajectory for a probe of a robotic body scanning system along at least a portion of a human body, the robotic body scanning system including the probe, a surface sensing system, and a robotic manipulator, the probe attached to the robotic manipulator, said method comprising:
receiving at least one task from a medical professional;
acquiring at least one surface profile of the portion of the human body with the surface sensing system;
identifying at least one planned trajectory based on the at least one task and the at least one surface profile;
adapting the at least one planned trajectory to an adapted trajectory based on the at least one surface profile; and
sending the adapted trajectory to a motion controller.

20. The method in accordance with claim 19 further comprising approving the adapted trajectory by a medical professional.
